(19) United States
(12) Patent Application Publication
(10) Pub. No.: US 2012/0165771 A1
(43) Pub. Date: Jun. 28, 2012

(54) Absorbent Articles With Multiple Active Graphics

(75) Inventors: Marcille Faye Ruman, Oshkosh, WI (US); Shannon K. Melius, Appleton, WI (US); Mary L. Schwer, Neenah, WI (US); Robert E. MacDonald, Kohler, WI (US); Eric Donald Johnson, Larsen, WI (US); Thomas Michael Ales, Neenah, WI (US); Jennifer Leigh Skabroud Misek, Neenah, WI (US); Terri Lynn Meisel, Appleton, WI (US); Tami L. Kurtz, Oshkosh, WI (US)

(73) Assignee: KIMBERLY-CLARK WORLDWIDE, INC., Neenah, WI (US)

(21) Appl. No.: 12/976,734

(22) Filed: Dec. 22, 2010

(51) Int. Cl. A61F 13/42

(52) U.S. Cl. 604/361; 604/385.01

(57) ABSTRACT

Absorbent articles are disclosed that have been printed with active graphics alone or in combination with permanent graphics. The active graphics may be used, for instance, as a wetness indicator on an absorbent article. In accordance with the present disclosure, the active graphics include at least one disappearing graphic and at least one appearing graphic. The disappearing graphic and appearing graphic allow for complete scene changes on the article and also allow for the illusion of movement of objects in the scene.

### BACKGROUND

Many different types of absorbent products exist that are designed to be worn or otherwise associated with the body for absorbing body fluids. Such absorbent products can include, but are not limited to, diapers, training pants, adult incontinence products, feminine hygiene products, bed liners, bandages, and the like. In some embodiments, the absorbent articles contain a cover material, a liner, and an absorbent structure positioned in between the outer cover and the liner. The absorbent structure typically contains superabsorbent particles. Many absorbent articles, especially those sold under the trade name HUGGIES® by the Kimberly-Clark Corporation, are so efficient at absorbing liquids that it is sometimes difficult for the wearer or the caregiver to tell whether or not the absorbent article has been insulted with a body fluid, such as urine.

Accordingly, various types of moisture or wetness indicators have been suggested for use in absorbent articles. The wetness indicators can be used for different purposes. For instance, the wetness indicators can be designed to assist parents or attendants by helping them identify a wet diaper condition early on.

Wetness indicators can also be used as a training aid for small children during the toilet training process. For example, wetness indicators can be designed to discourage small children from wetting the absorbent article and to encourage small children to use a toilet like an adult.

Wetness indicators used in the past have included wetness indicator graphics that have been printed on the absorbent article. Absorbent articles having wetness indicator graphics, for instance, are described in U.S. Pat. No. 6,297,424, in U.S. Pat. No. 6,710,221, and in U.S. Patent Application Publication No. 2006/0149197, which are all incorporated herein by reference. Although the above patents have provided great advances in the art, further improvements are still needed.

For instance, prior commercial wetness indicator graphics have been created using water soluble inks that dissolve when wet. Thus, once contacted with urine, the graphics smear and fade indicating that the absorbent article is wet. Unfortunately, however, graphics produced with water soluble inks have been somewhat limited in size, shape, position, line width, color and the like in order to ensure sufficient fading when contacted with urine.

Such fading graphics have been found to be somewhat difficult to discern by either the child wearing the absorbent article or by a caregiver or attendant. Thus, a need exists for absorbent articles that include changing graphics which are more noticeable to the user and/or to the caregiver. In particular, a need exists for an absorbent article with changing graphics that are more engaging and have more appeal than many prior commercial products.

### SUMMARY

The present disclosure is generally directed to absorbent articles having active graphics. The active graphics are graphics that change or are activated when contacted with an activation agent. In one embodiment, for instance, the absorbent article includes at least one appearing graphic and at least one disappearing graphic. The active graphics can be designed to change when contacted with various activation agents. In one embodiment, for instance, the activation agent that causes the graphics to change may comprise a body exudate, such as urine or a component in urine. In this manner, the active graphics can be used as a wetness indicator. In addition to active graphics, the absorbent article may also include permanent graphics that together with the active graphics depict an overall scene. As will be described in greater detail below, the various embodiments of the present disclosure provide for an improved noticeable change in appearance when the absorbent article contacts an activation agent. In one embodiment, the present disclosure allows the wearer of the absorbent article or allows a caregiver to more readily notice when the absorbent article has been wetted.

Prior to describing the embodiments of the present disclosure in detail, the following are definitions of various terms.

The term “active graphic” as used herein refers to an appearing graphic, a disappearing graphic, a color changing graphic or a combination thereof. The term “appearing graphic” is used herein to refer to a graphic that becomes visible (appears) or becomes significantly more visible when exposed to an activation agent. Conversely, the term “disappearing graphic” is used herein to refer to a graphic that becomes invisible (disappears) or significantly less visible when exposed to an activation agent, such as a body exudate, such as urine, fecal matter, a vaginal secretion or a nasal discharge.

In particular embodiments, the active graphic can comprise a fading graphic which is formed from an ink that is soluble in aqueous solutions such as a body exudate. The ink is positioned in the absorbent article so that it becomes wet and dissolves when the product is insulted with a liquid. Once dissolved, the ink washes away from the outer cover and is obscured by the outer cover. As a result, the active graphic seems to disappear from view.

Suitable urine-soluble inks are available from a variety of commercial vendors, such as Sun Chemical Corp. of Philadelphia, Pa., USA under the trade designation AQUA DESTRUCT. Particular urine-soluble compositions are disclosed in U.S. Pat. No. 4,022,211 issued May 10, 1977 to Timmons et al., which is incorporated herein by reference. The ink color can be selected to provide a pleasing appearance and graphic impact, including fading rapidly upon contact with liquid.

The active graphic can also comprise a color changing graphic which is formed from a composition such as an ink or adhesive that changes color when exposed to an aqueous solution such as urine. A color change composition can be adapted to blend in with a background or surrounding color, either before or after exposure to the aqueous solution or to undergo a more noticeable color change. Suitable compositions of this color-change type are available from a variety of commercial vendors, such as a pH-change/color-change hot melt adhesive available from Findley Adhesives, Inc. of Wauwatosa, Wis., USA. Alternatively, the active graphic can comprise pH sensitive inks, fugitive inks, colored absorbent particles, hydratable salts, moisture sensitive films, enzymes, heat sensitive inks and dyes, or the like.

In one embodiment, a color changing active graphic composition may comprise a matrix-forming component, a colorant, a surfactant and a pH adjuster. The matrix-forming component may be a water-insoluble, film-forming polymer or an ink base, such as a flexographic varnish having an organic solvent base. The colorant can be a pH indicator, such as a charged pH indicator, capable of changing color in response to the presence of a fluid. The surfactant may include a charged surfactant that attracts the colorant or a combination of charged surfactants that attract the colorant and a neutral surfactant. The pH adjuster may include a low molecular weight organic acid and a high molecular weight organic acid.

The matrix-forming component may comprise, for instance, an acrylate/acrylamide copolymer, a polyurethane adhesive, methylcellulose, and/or copolymers of vinylpyrrolidone and dimethylaminopropyl methacrylamide. Such color changing compositions are disclosed, for instance, in U.S. patent application Ser. Nos. 12/503,364 or 12/503,380, which are incorporated herein by reference.

In another embodiment, a color-changing composition is used that is water-resistant and water-insoluble. The color-changing composition can form thin films of various patterns and shapes on a substrate upon drying and the films can generate color upon contact with an aqueous medium. In one embodiment, the composition includes various components dissolved in a volatile organic solvent medium. The components can comprise a leuco dye or a combination of leuco dyes, an electron-withdrawing color developer or a combination of color developers that can form color complexes when associated with the leuco dye under the proper conditions, and a separator or combination of separators, which when dissolved in the system in an adequate quantity, can prevent the formation of the color complexes. In one embodiment, the components can be contained within a polymeric encapsulation matrix. The encapsulating matrix can contain at least one kind of polymeric resin that can form a thin film on substrate surfaces with good adhesion. In addition to the above, the solution may also contain various other additives to adjust physical properties.

Leuco dyes are generally referred to as colorless or pale-colored basic dyes, because the dye molecules can acquire two forms, one of which is colorless. Although not intended to be bound by theory, it is believed that the color-developer agent functions as a Lewis acid, which withdraws electrons from the leuco dye molecule to generate a conjugated system. Hence, the leuco dye appears to manifest color from an originally colorless state.

For example, the spiro form of an oxazine is a colorless leuco dye; the conjugated system of the oxazine and another aromatic part of the molecule is separated by an sp³-hybridized “spiro” carbon. After protonating a part of the molecule, irradiation with UV-light or introducing other kind of such change, the bond between the spiro carbon and the oxazine interrupts, the ring opens, the spiro carbon achieves sp² hybridization and becomes planar, the aromatic group rotates, aligns its π-orbitals with the rest of the molecule, and a conjugated system forms, with ability to absorb photons of visible light, and therefore appear colorful.

The leuco dyes that may be employed can be selected from a variety of dyes including, for example, phthalide leuco dyes, triarylmethane leuco dyes, and fluoran leuco dyes. Examples may include (1) Triarylmethane-based dyes, e.g. 3,3-bis(p-dimethylaminophenyl)-6-dimethylaminophthalide, 3,3-bis(p-dimethylaminophenyl)phthalide, 3-(p-dimethylaminophenyl)-3-(1,2-dimethylindol-3-yl)phthalide, 3-(p-dimethylaminophenyl)-3-(2-methylindol-3-yl)phthalide, 3,3-bis(1,2-dimethylindol-3-yl)-5-dimethylaminophthalide, 3,3-bis(1,2-dimethylindol-3-yl)-6-dimethylaminophthalide, 3,3-bis(9-ethylcarbazol-3-yl)-6-dimethylaminophthalide, 3,3-bis(2-phenylindol-3-yl)-6-dimethylaminophthalide, 3-p-dimethylaminophenyl-3-(1-methylpyrrol-3-yl)-6-dimethylanninophthalide, etc. (2) Diphenylmethane-based dyes, e.g., 4,4′-bisdimethylaminobenzhydryl benzyl ether, N-halophenylleucoauramine, N-2, 4,5-trichlorophenyl-leucoauramine, etc. (3) Lactam-based dyes, e.g., rhodamine-B-anilinolactam, rhodamine-(p-nitroanilino)lactam, rhodamine-(o-chloroanilino)lactam, etc. (4) Fluoran-based dyes, e.g., 3-dimethylamino-7-methoxyfluoran, 3-diethylamino-6-methoxyfluoran, 3-di-ethylamino-7-methoxyfluoran, 3-diethylamino-7-chlorofluoran, 3-diethylamino-6-methyl-7-chlorofluoran, 3-di-ethylamino-6,7-dimethylfluoran, 3-(N-ethyl-p-toluidino)-7-methylfluoran, 3-diethylamino-7-(N-acetyl-N-methylamino)fluoran, fluoran, 3-diethylamino-7-(N-methylamino)fluoran, 3-diethylamino-7-dibenzylaminofluoran, 3-diethylamino-7-(N-methyl-N-benzylamino)fluoran, 3-diethylamino-7-(N-chloroethyl-N-methylamino)fluoran, 3-diethylamino-7-N-diethylaminofluoran, 3-(N-ethyl-p-toluidino)-6-methyl-7-phenylaminofluoran, 3-(N-ethyl-p-toluidino)-6-methyl-7-(p-toluidino)fluoran, 3-diethylamino-6-methyl-7-phenylaminofluoran, 3-dibutylamino-6-methyl-7-phenylaminofluoran, 3-diethylamino-7-(2-carbomethoxyphenylamino)fluoran, 3-(N-cyclohexyl-N-methylamino)-6-methyl-7-phenylaminofluoran, 3-pyrrolidino-6-methyl-7-phenylaminofluoran, 3-piperidino-6-methyl-7-phenylaminofluoran, 3-diethylamino-6-methyl-7-(2,4-dimethylamino)fluoran, 3-diethylamino-7-(o-chlorophenylamino)fluoran, 3-dibutylamino-7-(o-chlorophenylamino)fluoran, 3-pyrrolidino-6-methyl-7-(p-butylphenylamino)fluoran, 3-(N-methyl-N-n-amylamino)-6-methyl-7-phenylaminofluoran, 3-(N-ethyl-N-n-amylamino)-6-methyl-7-phenylaminofluoran, 3-(N-ethyl-N-isoamylamino)-6-methyl-7-phenylaminofuluoran, 3-(N-methyl-N-n-hexylamino)-6-methyl-7-phenylaminofluoran, 3-(N-ethyl-N-n-hexylamino)-6-methyl-7-phenylaminofluoran, 3-(N-ethyl-N-β-ethylhexylamino)-6-methyl-7-phenylaminofluoran, etc. The basic dyes useful in this invention are not limited to those exemplified above, and at least two of them can be used in admixture.

The separator or desensitizer can be any of known component agent which exhibit good solubility in both water and organic solvents. Generally, the separators are preferred to be neutral molecules that are without a charge, such as polyalkylene glycol of <1000 Daltons, polyalkylene oxide of <10000 Daltons, block copolymers of polyoxyethylene polyoxypropylene glycol, polyoxyethylene nonylphenyl ether, polyoxyethylene distyrenated phenyl ether, neutral surfactants. Other examples of such separators may include glycerin; dodecylamine; 2,4,4-trimethyl-2-oxazoline; polyolefin glycols such as polyethylene glycol, polypropylene glycol and copolymer of ethylene glycol and propylene glycol; polyoxyethylene lauryl ether, polyoxyethylene oleyl ether, polyoxyethylene nonyl phenyl ether, polyoxyethylene sorbitan monolaurate, polyethylene glycol monostearate.

Generally, the color-developers exhibit good solubility in organic solvents. Examples of suitable developers include bisphenol A, zinc chloride, zinc salicylate, and phenol resins. Other examples of color developing materials to be used conjointly with the lecuo dyes may include: 4-tert-butylphenol, α-naphthol, β-naphthol, 4-acetylphenol, 4-tert-octylphenol, 4,4′-sec-butylidenephenol, 4-phenylphenol, 4,4′-dihydroxydiphenylmethane, 4,4′-isopropylidene diphenol, hydroquinone, 4,4′-cyclohexylidene diphenol, 4,4-dihydroxy diphenylsulfide, 4,4′-thiobis(6-tert-butyl-3-methylphenol), 4,4′-dihydroxydiphenyl sulfone, hydroquinone monobenzyl ether, 4-hydroxybenzophenone, 2,4-dihydroxybenzophenone, 2,4,4′-trihydroxybenzophenone, 2,2′,4,4′-tetrahydroxybenzophenone, dimethyl 4-hydroxyphthalate, methyl 4-hydroxybenzoate, ethyl 4-hydroxybenzoate, propyl 4-hydroxybenzoate, sec-butyl 4-hydroxybenzoate, pentyl 4-hydroxybenzoate, phenyl 4-hydroxybenzoate, benzyl 4-hydroxybenzoate, tolyl 4-hydroxybenzoate, chlorophenyl 4-hydroxybenzoate, phenylpropyl 4-hydroxybenzoate, phenethyl 4-hydroxybenzoate, p-chlorobenzyl 4-hydroxybenzoate, p-methoxybenzyl 4-hydroxybenzoate, novolak type phenol resins, phenol polymers and like phenol compounds.

Examples of suitable encapsulating polymers may include, for instance, Gantrez series polymers from International Specialty Products, Inc., Dermacryl 97 from National Starch, and Amphomers from Akzo Nobel. The encapsulation matrix may also be a mixture of various chemicals dissolved in an organic solvent system. Examples of such a system may include organic solvent-based varnishes, such as varnishes made by Sunchemical Co. Examples of polymers and copolymers that are substantially soluble in organic solvents may include: styrene-butadiene copolymers, acrylic acid ester polymers, polyvinyl acetates, polyvinyl chlorides; polyvinylbutyral, polyvinyl acetate, vinyl chloride-vinyl acetate copolymer, acrylic resin, styrene resin, polyester resin, and polyvinyl acetate, vinyl chloridevinyl acetate copolymer, styrene-maleic anhydride copolymer, isobutylene-maleic anhydride copolymer, polyvinyl butyral.

The above composition also contains a volatile organic solvent system. Examples of such volatile organic solvents or a mixing solvent system may include low-molecular weight alcohols, such as butanol, ethanol, propanol, and acetone or tetrahydrofuran or their mixtures.

In addition to ink compositions (which includes dyes), the active graphic can also comprise an adhesive, or any other suitable chemical.

In contrast to active graphics, the term “permanent graphic” is used herein to refer to a graphic that does not substantially change its degree of visibility when the absorbent article is insulted with urine in simulated use conditions. The change in visibility of a graphic or a portion of a graphic can be determined based on a person's observation of the graphic before and after the article containing the graphic is exposed to liquid. For purposes hereof, an article is exposed to liquid by immersing the article completely in an aqueous solution containing 0.9 weight percent sodium chloride, used at room temperature (≅23° C.), for a period of twenty minutes. After 20 minutes the product is removed from the aqueous solution and placed on a TEFLON™ coated fiberglass screen having 0.25 inch openings, which is commercially available from Taconic Plastics Inc., Petersberg, N.Y., USA, which in turn is placed on a vacuum box and covered with a flexible rubber dam material. A vacuum of 3.5 kilopascals (0.5 pounds per square inch) is drawn in the vacuum box for a period of 5 minutes, after which the article is removed and observed. The person with normal or corrected vision of 20-20 should make the observations from a distance of 1 meter in an environment providing 30 footcandles (320 Lux) of illumination. Changes in the visibility of the graphic should be identified, and distinguished where necessary from changes in the color of other materials such as fluff pulp within an absorbent assembly. Desirably, the permanent graphic can be configured so that the entire graphic also does not substantially change its appearance, size or shape when the product is insulted with liquid or exposed to the environment.

The graphics of the absorbent article can be constructed to provide a story line involving a permanent character graphic and an active object graphic. The term “character graphic” is used herein to refer to a graphic containing an anthropomorphous image, and in particular an image having or suggesting human form or appearance which ascribes human motivations, characteristics or behavior to inanimate objects, animals, natural phenomena, cartoon characters, or the like. The character graphics can comprise permanent graphics, active graphics, or both permanent and active graphics.

The character graphic can desirably comprise a portion of the entire absorbent article graphic that sets up a theme for the illustrated scene. As such, the character graphic can provide an opportunity for educational interaction between the child and the parent or caregiver. More specifically, the parent or caregiver can use the graphic story-line to make up a game or story for the purpose of toilet training progress.

Suitable character graphics can include animals, people, inanimate objects, natural phenomena, cartoon characters, or the like that can or can not be provided with human features such as arms, legs, facial features or the like. For purposes of enhanced toilet training, it may be desirable for the character graphic to be familiar to the child, such as an identifiable cartoon character. The character graphics should at least be a type that the child can relate to, examples of which could include animals, toys, licensed characters, or the like. Character graphics can be made more personable and friendly to the child by including human-Like features, human-like expressions, apparel, abilities, or the like. By way of illustration, an animal character graphic can be shown smiling, wearing clothing, playing sports, fishing, driving, playing with toys, or the like. In particular embodiments, the character graphic can desirably be created to project an appearance that could be described as friendly, positive, non-intimidating, silly, independent, inspirational, active, expressive, dauntless and/or persevering. Characters and other elements can be selected for use with an absorbent article in order to enhance the experience and improve the aesthetic qualities of the article. Graphics comprising themes, characters and other elements can also be designed to focus on a particular gender. For example, commercial print processes, product construction materials, graphical themes, and active graphics can be selected to focus either on a boy/male gender or on a girl/female gender.

In general, absorbent articles made in accordance with the present disclosure contain graphics comprising multiple objects such as various images and/or characters. All of the graphics can be assembled together on the absorbent article so as to present an integrated look that depicts a scene. In addition to characters and other images as described above, the scene can include outlines, a background, a foreground, and one or more silhouettes.

An “outline” refers to the lines by which the essential features or main aspects of an image, object or character is defined or bound. In one embodiment, for instance, a character appearing on an absorbent article may include an outline made from permanent graphics or from white space. Within the outline, the character may be colored using active graphics.

As used herein, the “background” of a scene is the surface against which represented objects and forms are perceived or depicted. The background is situated behind the location of an image or object. Each scene includes a background. In addition, various elements within the scene may also include a background.

As used herein, the “foreground” describes the location of an image or object which is situated in front of something. As understood by one skilled in the art, an object may be both in the foreground and the background.

A “silhouette” is a representation of the general shape of an object, image or character without the essential features or main aspects of the object filled in. The silhouette, for instance, excludes the graphic details or elements intended to show the dimensionality or recognition of the image or character such as facial features, clothing details, flower petals, and the like. A silhouette may also comprise a dark image outlined against a lighter background or vice versa.

The present disclosure, as described above, can be directed to applying graphics to an absorbent article including a surface, such as an inside or outside surface. The surface can have a “printable surface area” which is defined as the surface area of a rectangle which has a length measured from the front edge of the absorbent article to the back edge of the absorbent article and has a width measured as the narrowest portion of the surface, such as the outer cover, which is usually within the crotch region. It should be understood that the permanent and active graphics contained on an absorbent article may have a width that is greater than the width of the printable surface area as defined.

As used herein, the “active graphic surface area” refers to the surface area upon which the active graphic composition has been applied. The “active graphic perimeter surface area”, on the other hand, refers to the area defined by the perimeter of the outer most elements of the active graphic and may include areas within the perimeter where an active graphic composition has not been applied.

The outer cover can also include “white space”. White space is defined as the predominate color of the surface of the absorbent article. The white space, for instance, may be the color of the material used to create a surface of the absorbent article prior to being treated with any printed graphics. For example, the white space may comprise the natural color of the material used to form a surface of the absorbent article. In one embodiment, the white space present on the absorbent article is white in color and has a white appearance. The white space can be formed from a non-printed area on a substrate or can comprise an area that has been printed or treated with a pigment or ink if it is the predominant color. In one embodiment of the present disclosure, the active graphics fade, disappear, turn clear, or turn the same color as the white space which increases the white space on the outer cover. Alternatively, the active graphics can turn from the white space color to another color thus decreasing the white space on the outer cover.

In one embodiment, the present disclosure is directed to an absorbent article comprising an outer cover having an interior surface and an exterior surface. An absorbent structure is positioned adjacent to the interior surface of the outer cover. In one embodiment, the absorbent article can further include a liquid permeable liner. The absorbent structure can be positioned in between the outer cover and the liquid permeable liner. White space is also formed from materials under the outer cover; these materials (e.g., fluff/SAM of absorbent system) create a non colored “white” surface that can reflect incident light and create a more opaque/solid white appearance for one viewing the product from exterior. The naturally occurring color may be white, off-white, yellowish, etc. depending on whether whitening agents or other treatments are added to the internal absorbent article components.

In accordance with the present disclosure, graphics are incorporated into the absorbent article such that the graphics are visible from a surface of the article. The graphics, for instance, can be visible from the inside surface of the article (i.e. the surface that faces the wearer) or from an outside surface of the article. The graphics can depict a scene on the absorbent article. The graphics include a first active graphic and a second active graphic. The first active graphic comprises a disappearing graphic, while the second active graphic comprises an appearing graphic. The disappearing graphic and the appearing graphic, for instance, may undergo a change when contacted with an activation agent, such as a body fluid.

Having a disappearing graphic appear in combination with an appearing graphic can offer various benefits and advantages. For example, when the graphics are contacted with an activation agent, various images in a scene may disappear, while other images may simultaneously or over time appear. Such a change in the appearance of the absorbent article can be dramatic and very noticeable to the wearer and/or to the caregiver.

In one embodiment, for instance, the graphics on the absorbent article depict an object, such as a character. An “object” as used herein generally refers to any image created by the graphics. The first active graphic and the second active graphic may cause the object to change into a different object when contacted with the activation agent. For example, the active graphics may cause a character to change into a different character when the graphics are contacted by an activation agent. In another embodiment, the graphics may include a background scene and/or a foreground scene. The appearing graphics and disappearing graphics can be used to cause the background scene to change into a different background scene and/or can cause the foreground scene to change into a different foreground scene. The background scene and/or the foreground scene can also change in combination with changes to one or more focal images in the scene, such as a character.

In one embodiment, the disappearing graphics and the appearing graphics may work in conjunction to provide an illusion of movement of an object in the scene. For instance, the first active graphic may depict a first image and the second active graphic may depict a second image. The first image and the second image may be of the same object in the scene at different positions. Thus, when the first image disappears and the second image appears, the object appears to move. The object, for instance, may be a ball, a toy, or an animal. The object may also be a character or a portion of a character, such as a character's arm. In yet another embodiment, the object may be a vehicle, such as a car, a truck, an airplane, a bicycle, a motorcycle, or the like.

In one embodiment, the graphics may include more than one disappearing graphic and more than one appearing graphic so that multiple objects appear to move in the scene when the absorbent article is contacted with an activation agent.

In general, the disappearing graphics and the appearing graphics can overlap on the absorbent article or be in a non-overlapping relationship. In one embodiment, the active graphics also have a relatively large size to ensure that the graphics can be noticed.

In accordance with the present disclosure, in one embodiment, the at least one active graphic can have a surface area of greater than about 800 mm², such as greater than about 1000 mm². In one embodiment, one or more active graphics can occupy all of the surface area of the outer cover, such as the entire printable surface area of the outer cover. The active graphic in various embodiments may have a surface area of less than about 140,000 mm², such as less than about 45,000 mm², such as less than about 20,000 mm², such as less than about 8000 mm² depending on the type and size of the article (adult article versus child article).

As described above, the outer cover may define a printable surface area. In one embodiment, the active graphic may occupy greater than 1% of the printable surface area of the outer cover, such as greater than about 1.2%, such as greater than about 1.4% of the printable surface area of the outer cover.

The absorbent article can generally be divided into three regions. For instance, the absorbent article can include a front region, a back region, and a crotch region in between the front region and the back region. In one particular embodiment, each of the regions can have substantially the same length in the longitudinal direction. The active graphics can be primarily located in the crotch region or completely located in the crotch region. As used herein, the term “primarily” means that more than 50% of the outline be located in the crotch region.

In an alternative embodiment, the active graphics can be primarily located in the front region or completely located in the front region. In general, the active graphics can be positioned at any suitable location on the absorbent article. The location of various active graphics on absorbent articles, for instance, are shown and/or described in U.S. Pending Application Publication No. 2006/0149197 or in U.S. Patent Application Publication No. 2009/0062757, which are incorporated herein by reference.

The outside surface or the inside surface of the absorbent article may also be divided into two halves in the longitudinal direction. For instance, the absorbent article can include a front half and a back half. The front half extends from the front edge of the article to the midpoint of the article between the front edge and the back edge. The back half of the absorbent article, on the other hand, may extend from the back edge of the article to the midpoint. The front half and the back half can each include a printable surface area that is generally one half of the printable surface area of the entire outside surface or inside surface. In many applications, the active graphics and/or permanent graphics can all be present only on the front half or only on the back half of the absorbent article.

In one embodiment, the absorbent article can include a waist opening opposite the midpoint of the article and the disappearing active graphics may be positioned closer to the midpoint in relation to the appearing active graphics. In this manner, the disappearing graphics may disappear before the appearing graphics appear, especially if the activation agent is a body fluid, such as urine. For instance, as the urine migrates through the absorbent article, the urine will first contact the disappearing graphics and then contact the appearing graphics. Of course, depending upon the desired result, the order or position of the active graphics may also be reversed.

The manner in which the active graphics and/or permanent graphics are incorporated into the absorbent article can vary depending upon the particular application. In one embodiment, for instance, the active graphics can be disposed on the interior surface of the outer cover, while the permanent graphics can be disposed on the exterior surface of the outer cover. Placing the active graphics on the interior surface of the outer cover ensures that the active graphics come into contact with urine when the absorbent article is insulted. In one embodiment, the outer cover may include a liquid permeable outer layer and a liquid impermeable inner layer. The at least one active graphic can be disposed on the surface of the inner layer that forms the interior surface of the outer cover. Permanent graphics, on the other hand, may be disposed on the opposite surface of the inner layer or on the same surface.

Other features and aspects of the present disclosure are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIGS. 1, 2 and 3 are plan views of one embodiment of an absorbent article made in accordance with the present disclosure including active graphics;

FIGS. 4 and 5 are plan views of an alternative embodiment of an absorbent article in accordance with the present disclosure;

FIGS. 6 and 7 are plan views of an alternative embodiment of an absorbent article in accordance with the present disclosure; and

FIGS. 8 and 9 are plan views of another alternative embodiment of an absorbent article in accordance with the present disclosure.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to absorbent articles incorporating multiple active graphics alone or in combination with permanent graphics. The active graphics can include, for instance, a combination of disappearing graphics, appearing graphics, and/or color-changing graphics. The use of disappearing and appearing graphics together in an absorbent article provides various benefits and advantages. For instance, the appearing and disappearing graphics can be used to produce the illusion of motion of an object on the absorbent article. In addition, appearing and disappearing graphics may be used in combination to change a complete scene on the absorbent article, a character on the absorbent article, the background scene on the absorbent article, the foreground scene on the absorbent article or combinations thereof. As used herein, a complete scene change occurs when the focal images in the scene change in conjunction with changes in the background and/or the foreground. For example, if the focal image of a graphic is a character, a complete scene change occurs when the character changes into a different character or changes expression and appearance in conjunction with other changes in the scenery.

The active graphics used in the present disclosure can undergo a change when contacted with an activation agent. The activation agent can vary depending upon the particular application and the desired result. Activation agents that may be used include, for instance, pH change, temperature change, contact with an analyte, contact with a microbial load, or even contact with air. In one embodiment, the activation agent may comprise a body exudate, such as urine. For instance, if desired, the active graphics may be used as a wetness indicator. When used as a wetness indicator, the active graphics may not only be used to signal a caregiver that the absorbent article has been wetted, but may also be used as a tool for toilet training.

In one embodiment, the graphics appearing on the absorbent article not only include a combination of appearing graphics, disappearing graphics, and/or color changing graphics, but may also include a combination of active graphics and permanent graphics. For example, the active graphics and permanent graphics may be interrelated to display an overall scene with a common storyline. The permanent graphics may be used to accentuate the active graphics for providing a very prominent change when the absorbent article is wetted.

In one embodiment, for instance, the scene depicted upon the absorbent article provides an integrated, one-piece look that can be consistent with a background color that covers substantially the entire outer cover and side panels. The absorbent article can include graphics that, in one embodiment, eliminate a substantial amount of white area in the crotch region of the absorbent article and highlights the location of the active graphics when the product is dry. When contacted with an activation agent, the active graphics offer a dramatic visual change without compromising the integrated appearance of the product. The active graphics of the present disclosure can be incorporated into all sizes of absorbent articles and can be designed to appeal to a certain gender.

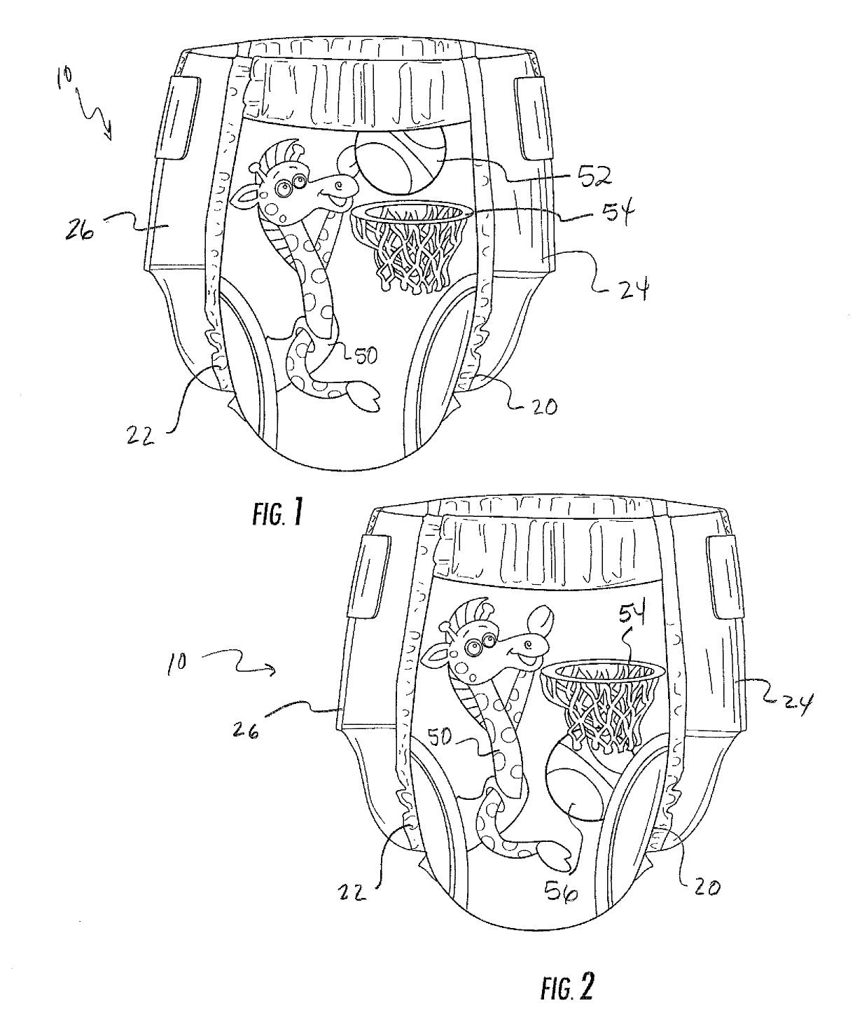

Referring to FIGS. 1 through 3, for instance, one embodiment of an absorbent article 10 made in accordance with the present disclosure is shown. In the figures, a child's training pant is generally shown. It should be understood, however, that the inventive concepts described herein can be applied to any suitable absorbent article, such as a diaper, an adult incontinence product, a feminine hygiene product or the like.

In the figures, the active graphics and permanent graphics are shown to be visible from an outside surface of the absorbent article, such as by being applied to the outer cover. It should be understood, however, that the graphics may also be applied so as to be visible from an inside surface of the article. For instance, when applied to a feminine hygiene product, the graphics may more appropriately be placed to be visible from the inside surface which is adjacent to the body of the wearer. In order to be visible from the inside surface, the active graphics may be applied to a liquid permeable bodyside liner, to a surge layer, to a portion of the absorbent core, or even to the outer cover material in certain embodiments.

Referring to FIG. 3, absorbent articles generally include an outer cover 12 that includes an exterior surface and an interior surface. Located adjacent the interior surface is an absorbent structure 15. Optionally, the absorbent article can also include a liquid permeable inner lining 14. The absorbent structure can be placed in between the outer cover 12 and the inner lining 14. The absorbent article 10 can further include elastic waistbands 16 and 18 and elastic leg members 20 and 22.

The absorbent article 10 as shown in FIG. 1 can be made from various materials. The outer cover 12 may be made from a material that is substantially liquid impermeable, and can be elastic, stretchable or nonstretchable. The outer cover 12 can be a single layer of liquid impermeable material, or may include a multi-layered laminate structure in which at least one of the layers is liquid impermeable. For instance, the outer cover 12 can include a liquid permeable outer layer and a liquid impermeable inner layer, such as a translucent or transparent film, that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent structure is positioned in between the outer cover and a liquid permeable bodyside liner 14. The bodyside liner 14 is suitably compliant, soft feeling, and non-irritating to the wearer's skin. The bodyside liner 14 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 14. For example, the bodyside liner can be made from a meltblown or spunbonded web of polyolefin fibers. The bodyside liner can also be a bonded-carded web composed of natural and/or synthetic fibers.

A suitable liquid permeable bodyside liner 14 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent structure, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference for all purposes.

It is also contemplated that elastomeric absorbent web structures may be used. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

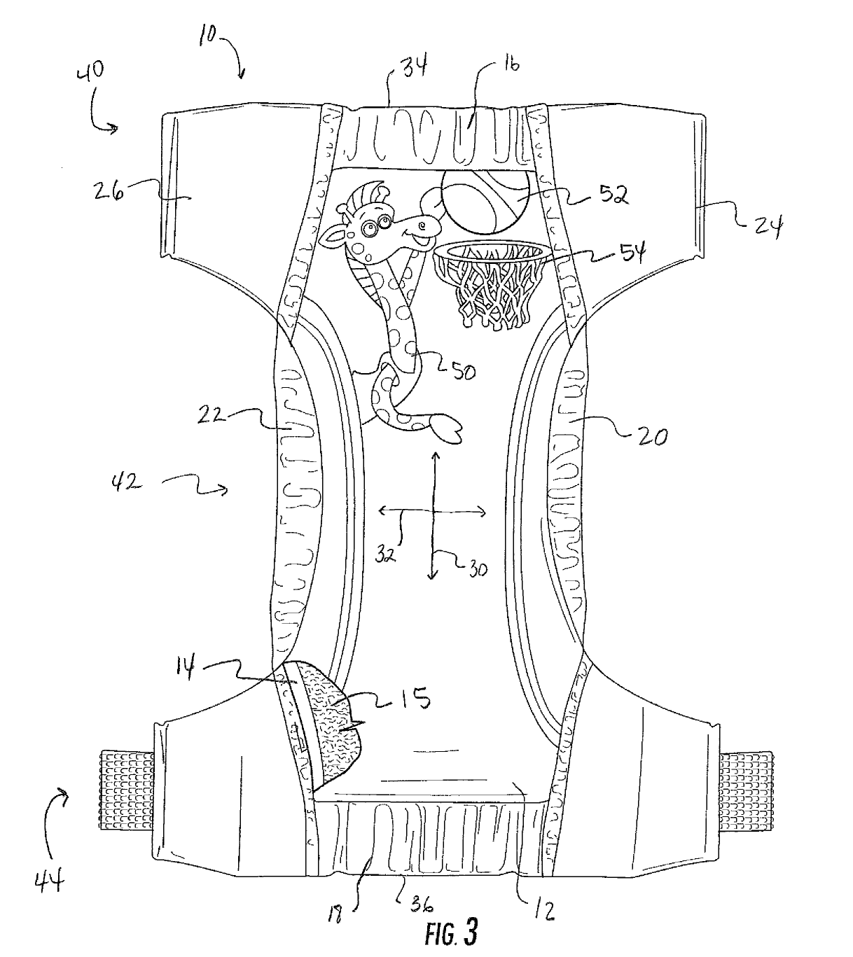

As shown in FIGS. 1 and 2, the absorbent article 10 can include side panels 24 and 26. The side panels 24 and 26 can have a color that blends with the overall scene appearing on the absorbent article. The side panels 24 and 26 can be permanently bonded together or can be releasably attached to one another. In FIG. 3, for instance, the side panels 24 and 26 are shown in an unattached state. In general, the side panels 24 and 26 are made from an elastic material, such as an elastic laminate.

As shown particularly in FIG. 3, the absorbent article 10 defines a longitudinal center line 30, a transverse center line 32, a first or front longitudinal end edge 34, and a second or back longitudinal end edge 36 which form a waist opening. The longitudinal axis lies in the plane of the article and is generally parallel to a vertical plane that bisects a standing wearer into left and right body halves when the article is worn. The transverse axis lies in the plane of the article generally perpendicular to the longitudinal axis.

In general, the absorbent article along the longitudinal axis can be divided into a front region 40, a back region 44, and a crotch region 42 positioned in between the front region and the back region. In one embodiment, the front region, the crotch region, and the back region can all have approximately the same length in the longitudinal direction.

The absorbent article can also be divided into a front half and a back half. The front half, for instance, may extend in the longitudinal direction from the front edge to the midpoint of the product, while the back half may extend from the back edge to the midpoint of the product.

As shown in FIGS. 1 through 3, the absorbent article 10 includes various graphics. In the embodiment illustrated, the scene displayed on the outer cover of the absorbent article includes a combination of permanent graphics and active graphics. As shown, the scene depicts a primary focal image which is a giraffe character 50. The giraffe character 50 is shown holding a basketball 52. More particularly, the giraffe character 50 is shown attempting to dunk the basketball 52 into a basket 54. Not shown, the multiple images can be made from multiple color compositions for further accentuating the scene and increasing its appeal.

As described above, the scene shown on the absorbent article 10 includes permanent graphics and active graphics. In one embodiment, for instance, the giraffe character 50 and the basketball net 54 may both be made from permanent graphics. In accordance with the present disclosure, the basketball 52, on the other hand, may comprise an active graphic, particularly a disappearing graphic. The basketball 52, for instance, may be configured to disappear when contacted with an activation agent.

Referring to FIG. 2, the scene further includes another active graphic 56. The active graphic 56 is also an image of a basketball. The active graphic 56 comprises an appearing graphic that is configured to appear once the active graphic contacts an activation agent.

Thus, in accordance with the present disclosure, the scene depicted upon the absorbent article 10 includes at least one disappearing graphic, at least one appearing graphic, and/or at least one color changing graphic. Once contacted with an activation agent, for instance, the active graphic 52 or basketball disappears, while the active graphic 56 or basketball appears. In this manner, the basketball appears to move in the scene providing an illusion of motion to a bystander. Specifically, the basketball appears to be dunked into the basketball net 54 by the giraffe 50.

In one embodiment, the activation agent that causes the active graphic 52 to disappear and the active graphic 56 to appear may be a body exudate, such as urine. Thus, the dunking of the basketball may indicate to the wearer or a caregiver that the absorbent article has been wetted. Due to the illusion of motion, the change in appearance of the article is easily noticeable.

When urine is the activation agent, the absorbent article may be designed to ensure that the active graphics are contacted with the urine when the absorbent article is wetted. In one embodiment, for instance, the active graphics may be positioned within or closer to the crotch region. A tradeoff may take place, for instance, between positioning the active graphics at least partially within the crotch region while still placing the active graphics at a location that are easily visible.

In another embodiment, the absorbent article may include a wicking layer that immediately wicks urine from the place of insult to the active graphics. For instance, a wicking strip may be placed in the article that contacts each of the active graphics. The wicking layer may be comprised of any suitable material capable of wicking liquids through the absorbent article. Wicking layers, for instance, are disclosed in U.S. Patent Application Publication No. 2008/0147030 and in U.S. Patent Application Publication No. 2009/0062757, which are incorporated herein by reference. The wicking layer, for instance, may comprise a tissue layer, a nonwoven layer, cellulosic materials, treated nonwovens, synthetic fibers, natural fibers, open celled foam, or the like. Various materials can be chosen so as to have an appropriate wicking speed.

In the embodiment illustrated in FIGS. 1 through 3, the disappearing graphic 52 is positioned farther away from the crotch region or the transverse center line 32 than the appearing graphic 56. In other embodiments, however, the scene may be changed in order to reverse the position of the active graphics. For example, if urine or a component in urine is the activation agent, it may be desirable for the disappearing graphic to disappear before the appearing graphic appears to better provide an illusion of motion of the object. Thus, in an alternative embodiment, the absorbent article may depict a scene in which the object undergoing motion includes an appearing graphic that is positioned farther away from the transverse center line 32 and closer to the waist opening of the absorbent article in relation to the disappearing graphic.

For example, in an alternative embodiment, the basketball 52 as shown in FIG. 1 may be placed adjacent to the opposite arm of the giraffe 50 and below the basket 54. The appearing graphic 56, on the other hand, may be positioned above the basket as shown in FIG. 1. Thus, in this embodiment, the disappearing graphic is positioned closer to the transverse center line 32 than the appearing graphic. When the absorbent article is wetted, the disappearing graphic will contact the body fluid before the appearing graphic as the liquid wicks through the absorbent article. Thus, in this embodiment, the giraffe 50 will appear to be shooting the basketball into the basket as opposed to dunking the basketball.

It should be understood, however, that in other embodiments it may be preferable to place the appearing graphic closer to the transverse center line as opposed to the disappearing graphic. For instance, depending upon the scene that is created using the graphics, it may be preferred from an aesthetic viewpoint for a first graphic to appear prior to a second graphic disappearing.

In the embodiment illustrated in FIGS. 1 through 3, the scene depicted includes one disappearing graphic and one appearing graphic. It should be understood, however, that in other embodiments the scene may include multiple appearing graphics and/or multiple disappearing graphics. For instance, in an alternative embodiment, the arm of the giraffe 50 may appear to move as the ball 52 is dunked through the basket 54. In particular, the arm of the giraffe as shown in FIG. 1 may comprise a disappearing graphic. The scene may further include an appearing graphic that comprises another arm of the giraffe in a different position. In another embodiment, the scene may include other appearing graphics that may appear as the ball is dunked through the basket 54. For instance, the scene may further include appearing graphics that comprise stars or rockets to further accentuate movement of the basketball.

In order to make the active graphics noticeable, the active graphics present on the absorbent article may have a relatively large size. For example, in one embodiment, the active graphics present within the scene may occupy greater than 1% of the printable surface area of the outer cover. For instance, the active graphics may occupy greater than 1.2% or greater than 1.4% of the printable surface area of the outer cover. The active graphics occupying the above proportions of the outer cover surface area is relatively large in comparison to many prior art products commercialized in the past.

In particular, the total surface area occupied by the active graphics may be greater than 800 mm², such as greater than 900 mm², such as greater than 1000 mm², such as even greater than 1200 mm².

The active graphics 52 and 56 as shown in FIGS. 1 and 2 can be produced using various different active graphic compositions. For instance, the disappearing graphic can be produced using a disappearing ink composition, while the appearing graphic can be produced using an appearing ink composition. The particular compositions used to produce the graphics may depend upon various factors, including the type of desired activation agent required for the change.

In one embodiment, the disappearing graphic may comprise a water-soluble ink composition that disappears or fades when contacted with water. The appearing graphic, on the other hand, may be made from an ink composition that changes from clear to a color when contacted with water. Both the disappearing graphic and the appearing graphic can both be made from color-changing compositions. For example, the disappearing graphic may be made from a composition that changes color in a manner that causes the image to disappear. For instance, the composition may change to a color that is the same color as the background of the absorbent article. Similarly, the appearing graphic may be comprised of a color-changing composition that is not discernible or noticeable until the color change occurs. The initial color of the composition, for instance, may match or blend the background color of the scene and then change color to become noticeable.

The active graphics and permanent graphics used to form the scene as shown in FIGS. 1 and 2 can be applied to the absorbent article in different ways. In one embodiment, for instance, the outer cover 12 of the absorbent article includes multiple layers. The outer cover 12 may include, for instance, an inner water impermeable film and an outer water permeable layer that may comprise, for instance, a nonwoven layer. The inner film may be clear such that graphics printed on the inner film can be visible from the exterior surface of the outer cover. As can be appreciated, when the activation agent is a body fluid, the active graphics should be applied to the absorbent article such that they contact any bodily fluids that may be absorbed by the article. In this regard, the active graphics may be printed on the interior surface of the outer cover such as on the inner surface of the inner film. The permanent graphics, however, can be printed on other layers of the outer cover. The permanent graphics, for instance, can be printed on the exterior surface of the outer cover, or can be printed on any of the interior layers either on the side facing the wearer or on the side opposite the wearer. In one embodiment, for instance, the active graphics and the permanent graphics can all be printed on the same surface in a single pass or in a multi-pass printing process.

In accordance with the present disclosure, both disappearing graphics and appearing graphics may be printed or otherwise applied on the interior surface of the outer cover, such as on the film as described above, in a single pass or in multiple passes. If the appearing graphic and disappearing graphic overlap, for instance, separate passes may be needed in which multiple layers are created on the inside of the outer cover.

Alternatively, the appearing and disappearing graphics may be printed or applied on different sides of a single substrate that is used to produce the outer cover. In yet another embodiment, the appearing graphic and the disappearing graphic may be printed or applied on separate materials and registered during production of the absorbent article 10.

The compositions used to make the active graphics can be formulated in different ways to achieve different results. As described above, for instance, the ink compositions used to produce the active graphics can comprise water-soluble ink compositions, compositions that, when activated, go from clear to a color, compositions that go from a color to clear, and color-changing compositions. In one embodiment, conventional inks or pigments, such as permanent ink compositions, can be combined with the color-changing compositions to produce a desired effect.

For example, in one embodiment, a functional ink may be combined with a conventional ink to produce a certain color. Once the composition is contacted with an activation agent, a desired color change may occur. The permanent ink may be blended with the composition, for instance, so that the active graphic blends in with the background color on the absorbent article when contacted with the activation agent or prior to contact with the activation agent.

Combining active graphic compositions with permanent graphic compositions to produce desired color effects can occur in numerous ways. In one embodiment, for instance, the compositions may be combined together and then applied to a substrate, such as an absorbent article. In an alternative embodiment, however, the desired effect may occur by printing the compositions onto the substrate in an overlapping manner.

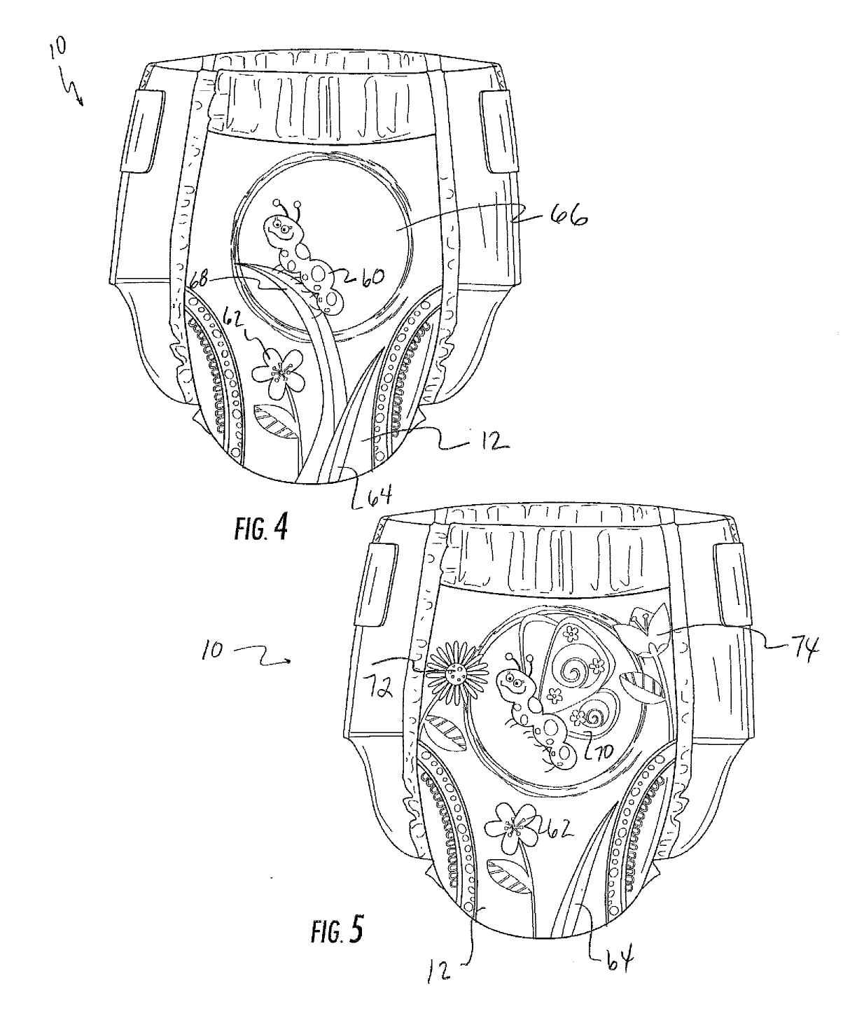

Referring now to FIGS. 4 and 5, another embodiment of an absorbent article 10 made in accordance with the present disclosure is shown. The absorbent article 10 includes an outer cover 12 that has been printed with permanent graphics and active graphics for creating a scene. The absorbent article 10 in FIG. 4 is intended to show the article prior to contact with an activation agent, such as urine. FIG. 5, however, shows various changes that can occur after the graphics come in contact with an activation agent.

As shown in FIG. 4, in this embodiment, the permanent graphics include a focal character 60 which, in this embodiment, comprises a caterpillar. The permanent graphics also include a flower 62, a leaf 64, and a sun 66.

When contacted with an activation agent, the caterpillar 60 turns into a butterfly as shown in FIG. 5. Various other images in the foreground and background also disappear and appear. For instance, as shown in FIG. 4, the scene includes a disappearing graphic 68 that comprises a leaf. In FIG. 5, multiple appearing graphics are included. The appearing graphics include butterfly wings 70, a flower 72, and another flower 74. As shown in FIG. 5, the leaf upon which the caterpillar 60 was resting in FIG. 4 disappears and the wings 70 appear creating the illusion that the caterpillar has turned into a butterfly and is now in flight. The flowers 72 and 74 also appear further enhancing the appeal of the scene and increasing the noticeability of the change.

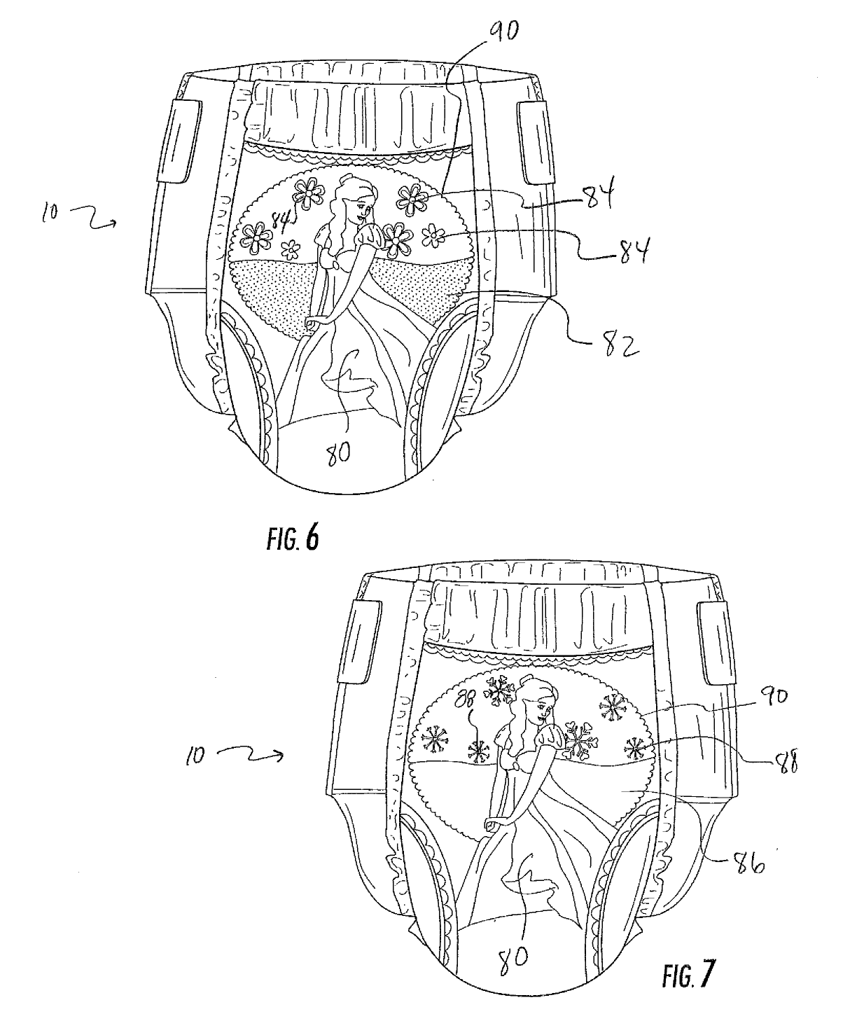

Referring to FIGS. 6 and 7, another embodiment of an absorbent article 10 made in accordance with the present disclosure is shown that also includes permanent graphics combined with active graphics. FIG. 6 is intended to show the absorbent article 10 prior to contact with an activation agent. FIG. 7, on the other hand, shows the absorbent article after contact with an activation agent. In this embodiment, the background scene changes when the article is brought into contact with an activation agent, such as a body fluid.

As shown in FIG. 6, the absorbent article 10 includes a main focal image which comprises a princess character 80. The princess character 80 is made from permanent graphics. Surrounding the princess character 80 are active graphics, namely a combination of appearing graphics and disappearing graphics. The disappearing graphics are shown in FIG. 6, while the appearing graphics are shown in FIG. 7.

The disappearing graphics include a grassy meadow image 82 and various flowers 84 which combine together to form a summer scene.

When the absorbent article 10 is contacted with an activation agent, however, the summer scene turns into a winter scene as shown in FIG. 7. In particular, the disappearing graphics 82 and 84 disappear while appearing graphics become visible. The appearing graphics include a snow field 86 and various snowflakes 88,

In the embodiment illustrated in FIGS. 6 and 7, the active graphics are all contained within an outline 90 made from permanent graphics. The outline 90 can be used to enhance the active graphics making the change from FIG. 6 to FIG. 7 more noticeable.

In FIGS. 6 and 7, the disappearing graphics and the appearing graphics overlap each other in the scene. Thus, as described above, the active graphic compositions may be applied in a multi-pass process on the same surface or on opposite surfaces of the outer cover. If the scene was altered so that the active graphics do not overlap, however, a single pass printing process may produce the entire background scene change.

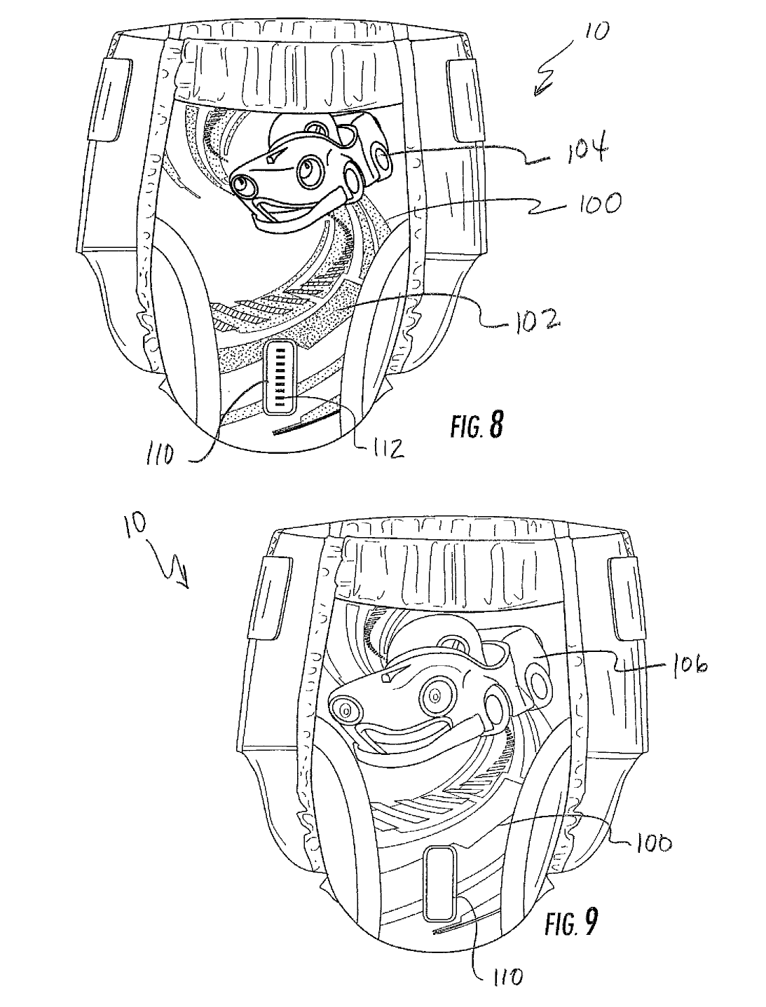

Referring to FIGS. 8 and 9, still another embodiment of an absorbent article made in accordance with the present disclosure is shown. FIG. 8 is intended to show the absorbent article 10 prior to contact with an activation agent, while FIG. 9 shows various changes that occur once an activation is present. The absorbent article 10 includes a combination of permanent graphics and active graphics. The active graphics include a combination of disappearing graphics and an appearing graphic.

In the embodiment illustrated in FIGS. 8 and 9, a car scene is depicted. In FIG. 8, the scene includes permanent graphics 100 which comprises the outline of a spiraling road or highway. The scene further includes a disappearing graphic 104 comprising a car character and disappearing graphics 102. The disappearing graphics 102 comprise one or more colors that are contained within the permanent graphics 100.

When contacted with an activation agent, the character graphic 104 disappears and the color graphic 102 disappears within the permanent graphic 100. As shown in FIG. 9, an appearing graphic 106 then becomes visible which comprises another image of the car character.

In this embodiment, when comparing FIG. 8 with FIG. 9, the car character appears to move due to the graphic 104 disappearing and the graphic 106 appearing. Further, the expression of the car character changes. In addition, the spiraling road changes from vibrant colors to only an outline as shown in FIG. 9.

The embodiment illustrated in FIGS. 6 and 7 and the embodiment illustrated in FIG. 9 are intended to provide wetness indicators that may discourage a child from wetting the absorbent article. In particular, a child may be taught to not wet their absorbent article or the princess in FIG. 6 will be taken from a warm summer scene into a winter scene. In FIGS. 8 and 9, the child can be instructed that wetting the garment may cause the car character to spin out of control.

Referring back to FIGS. 8 and 9, the absorbent article 10 further includes a gauge-like graphic 110 that includes gauge elements 112. The gauge elements 112 comprise disappearing graphics that disappear when contacted with an activation agent as shown in FIG. 9. The outline around the gauge elements, on the other hand, comprises permanent graphics. When used in conjunction with the car characters 104 and 106, the gauge-like graphic 110 may be used to further indicate that the absorbent article has come in contact with an activation agent, such as urine. By including disappearing graphics 112, the gauge-like graphic may indicate that the car character 106 has also run out of gas.

In the above examples, the active and permanent graphics were applied to the exterior surface of an absorbent article. It should be understood, however, that the above graphics can be applied equally so as to be visible from the interior surface of an absorbent article. In one embodiment, for instance, active graphics may be applied to both the outside surface and to the inside surface of the absorbent article.

When the active graphics are to be visible from an inside surface of the garment, the active graphics can be applied to a bodyside liner, a surge material, a wrap sheet that surrounds an absorbent structure, or may even be applied to the outer cover as long as the active graphics are visible from the interior.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An absorbent article comprising;
an outer cover having an interior surface and an exterior surface;
an absorbent structure positioned adjacent the interior surface of the outer cover; and
graphics incorporated into the absorbent article such that the graphics are visible from a surface of the absorbent article, the graphics depicting a scene on the absorbent article, the graphics including a first active graphic and a second active graphic, and wherein the first active graphic comprises a disappearing graphic and the second active graphic comprising an appearing graphic.

2. An absorbent article as defined in claim 1, wherein the first active graphic disappears and the second active graphic appears when contacted with an activation agent.

3. An absorbent article as defined in claim 2, wherein the absorbent article defines a transverse center line and wherein the first active graphic is positioned closer to the transverse center line than the second active graphic.

4. An absorbent article as defined in claim 2, wherein the absorbent article defines a transverse center line and wherein the second active graphic is positioned closer to the transverse center line than the first active graphic.

5. An absorbent article as defined in claim 2, wherein the activation agent comprises a body exudate.

6. An absorbent article as defined in claim 2, wherein the graphics depict an object and wherein the first active graphic and the second active graphic cause the object to change into a different object when the active graphics are contacted with the activation agent.

7. An absorbent article as defined in claim 6, wherein the object comprises a character.

8. An absorbent article as defined in claim 2, wherein the scene created by the graphics include a background scene and wherein the first active graphic and the second active graphic cause the background scene to change into a different background scene when the absorbent article is contacted with the activation agent.

9. An absorbent article as defined in claim 2, wherein the scene created by the graphics include a foreground scene and wherein the first active graphic and the second active graphic cause the foreground scene to change into a different foreground scene when the absorbent article is contacted with the activation agent.

10. An absorbent article as defined in claim 1, wherein the first active graphic and the second active graphic cause a complete scene change to occur when the active graphics are activated.

11. An absorbent article as defined in claim 1, wherein the first active graphic depicts a first image and the second active graphic depicts a second image, the first image and the second image being of an object in the scene and wherein, when the active graphics are activated and the first active graphic disappears and the second active graphic appears, an illusion of movement of the object occurs.

12. An absorbent article as defined in claim 11, wherein the object comprises a ball, a toy, a character, a vehicle, an animal, or a portion of any of the above.

13. An absorbent article as defined in claim 11, wherein the graphics further include a third active graphic comprising a disappearing graphic and a fourth active graphic comprising an appearing graphic and wherein the third active graphic depicts a third image and the fourth active graphic depicts a fourth image, the third image and the fourth image being of a second object in the scene and wherein, when the active graphics are activated, an illusion of movement of both objects occurs.

14. An absorbent article as defined in claim 1, wherein the first active graphic and the second active graphic are in an overlapping relationship.

15. An absorbent article as defined in claim 14, wherein the first active graphic and the second active graphic comprise at least a portion of a single character.

16. An absorbent article as defined in claim 15, wherein the first active graphic and the second active graphic cause an expression appearing on the character to change when contacted with an activation agent.

17. An absorbent article as defined in claim 1, wherein the first active graphic and the second active graphic are in a non-overlapping relationship.

18. An absorbent article as defined in claim 1, wherein the first active graphic is comprised of a color changing ink composition.

19. An absorbent article as defined in claim 1, wherein the second active graphic is comprised of a color changing ink composition.

20. An absorbent article as defined in claim 11, wherein the graphics further include a third active graphic that comprises a color changing graphic.

21. An absorbent article as defined in claim 13, wherein the third active graphic and the fourth active graphic are comprised of a color changing ink composition.

22. An absorbent article as defined in claim 1, wherein the first active graphic and the second active graphic are visible from an outside surface of the absorbent article.

23. An absorbent article as defined in claim 1, wherein the first active graphic and the second active graphic are visible from an inside surface of the absorbent article.

24. An absorbent article as defined in claim 1, wherein the first active graphic and the second active graphic are formed by applying an active graphic composition onto the outer cover of the absorbent article, the outer cover having a printable surface area, the active graphics occupying from about 1% to about 20% of the printable surface area of the outer cover.

25. An absorbent article as defined in claim 24, wherein all of the graphics present have a total surface area of at least 1000 mm².

26. An absorbent article comprising:
an outer cover and an absorbent structure positioned adjacent to the outer cover, the outer cover including a liquid impermeable layer having a first surface that faces the absorbent structure and a second surface that faces away from the absorbent structure; and
graphics incorporated into the absorbent article such that the graphics are visible on the absorbent article, the graphics including a first active graphic that comprises a first active graphic composition applied to the first surface of the outer cover and a second active graphic comprising a second active graphic composition also applied to the first surface of the outer cover, and wherein one of the active graphics comprises a disappearing graphic that changes from visible to invisible when contacted with an activation agent and wherein the other active graphic comprises an appearing graphic that changes from invisible to visible when contacted with the activation agent.

27. An absorbent article as defined in claim 26, wherein the outer cover comprises a liquid permeable outer layer and a liquid impermeable inner layer, the first active graphic composition and the second active graphic composition both being applied to the liquid impermeable inner layer on the first surface of the outer cover.

28. An absorbent article as defined in claim 26, wherein the absorbent article further includes permanent graphics, the permanent graphics being located on a surface of the liquid impermeable inner layer opposite the active graphics.

29. An absorbent article as defined in claim 27, wherein the absorbent article further includes permanent graphics, the permanent graphics being located on a surface of the liquid impermeable inner layer on the same side as the active graphics.

30. An absorbent article as defined in claim 26, wherein the absorbent article includes a crotch region and a waist opening and wherein the first active graphic depicts a first image and the second active graphic depicts a second image, the first image and the second image being of an object in the scene and wherein, when the active graphics are activated and the first active graphic disappears and the second active graphic appears, an illusion of movement of the object occurs, and wherein the second active graphic is located closer to the waist opening in relation to the first active graphic.

31. An absorbent article as defined in claim 26, wherein the second active graphic comprises an appearing graphic and wherein the second active graphic composition contains a permanent ink blended with an appearing ink.
